(19) United States
(12) Patent Application Publication
(10) Pub. No.: US 2006/0052690 A1
(43) Pub. Date: Mar. 9, 2006

(54) Contrast agent imaging-driven health care system and method

(76) Inventors: Saad Ahmed Sirohey, Pewaukee, WI (US); Gopal B. Avinash, New Berlin, WI (US); David Charles Mack, Waukesha, WI (US); Stanley Haim Fox, Brookfield, WI (US)

Correspondence Address: Patrick S. Yoder;FLETCHER YODER, P.O. Box 692289, Houston, TX, 77269-2289, US

(21) Appl. No.: 10/935,893

(22) Filed: Sep. 8, 2004

(51) Int. Cl. A61B 5/05

(52) U.S. Cl. 600/420

(57) ABSTRACT

Procedures for providing health care to a range of anatomies that require contrast agents for adequate imaging. Image data is acquired in accordance with a range of modalities, and at different times and on different patients, depending upon the particular implementation. The image data is processed in accordance with one of a range of manners and algorithms for a particular purpose and a specific tissue or organ. The resulting workflow paths provide novel combinations for rendering health care to specific tissues and organs by the use of contrast agent-based imaging.

### BACKGROUND

The invention relates generally to medical care, and particularly to medical care rendered based upon medical imaging, and more particularly to such care rendered based upon imaging created through the use of contrast agents to image body parts, tissues and anatomies for which such imaging and subsequent processing was heretofore unavailable.

A wide range of tissues may be imaged in a medical field through the use of various types of imaging systems. Over the past decades many such imaging systems have been developed and refined, including X-ray systems, which have moved from film-based systems to digital X-ray. Other important modalities include magnetic resonance imaging systems, computed tomography imaging systems, ultrasound systems, positron emission tomography systems, X-ray tomosynthesis systems, to mention only a few. In all of these, image data is acquired and stored for later processing and eventual image reconstruction. Modern techniques include analysis of image data both for image reconstruction and for evaluation of particular anatomies, tissues, structures, anomalies and disease states that may be visible or otherwise discernible from the image data. In a typical setting, reconstructed images are most often presented to a radiologist or other physician or clinician for use in rendering care.

While such systems provide excellent bases for health care, they have suffered from serious drawbacks, particularly relating to certain types of tissue, organs and tissue structures. Various soft tissues, for example, do not typically offer sufficient contrast to render structures in reconstructed images or do not sufficiently differentiate between tissues in such a way as to permit evaluation, processing and analysis of the image data. Many of these tissues are found in internal organs, such as the liver, kidneys, pancreas, vasculature, and so forth. While contrast agents have been employed for many years for certain types of imaging, such as X-ray imaging, certain techniques that have been developed for the imaging systems themselves, such as the imaging acquisition routines and image analysis protocols, have simply not been conjoined with the use of contrast agents so as to permit sufficient differentiation between the tissues or detailed analysis of such tissues.

There is a significant need in the art for improved procedures and workflows that allow the use of contrast agents in conjunction with certain types of tissues and anatomies so as to permit the use of sophisticated image reconstruction and processing to render improved health care. Such techniques and improvements would facilitate an entirely new field of health care in the case of organs and tissues that has simply been unavailable either with the use of contrast agents or without.

### BRIEF DESCRIPTION

The present invention provides novel techniques for rendering health care with respect to specific tissues and organs through the use of contrast agent-based imaging. The techniques employ contrast agents which are directed to the specific tissues or organs, and which may be administered in any one of several conventional manners. Imaging is then performed at one or multiple phases of progression of the contrast agent through the tissues. Imaging techniques may make use, for example, of any one of several available imaging modalities, or several modalities. As the contrast agent progresses through the tissues, rapid acquisition of image data allows for detection of anomalies and disease states, and for differentiation between specific types of tissue. Processing of the image data may be performed in accordance with sophisticated segmentation, visualization, and other data analysis techniques. The image data may include data from single-phase scans, multi-phase scans, scans or acquisitions at different points in time, profusion data, multi-modal data and multi-patient data. The data and processing thus permit analysis of different tissues in a single patient and progression of developments over time, as well as comparison of different types of tissue, including organ sizes and so forth between patients.

Among the types of analysis and processing that may be performed, the present techniques contemplate segmentation, sizing, visualization, registration, shape-based analysis, delineation, volumetric analysis, modeling and surgical navigation. Certain of these techniques may be performed in real-time, permitting active interfacing between a care provider and a patient. Other of the techniques may be performed retrospectively, including on single data sets and multiple data sets for different modality systems, different patients, and different times for a single patient.

The enhanced image data acquisition techniques and analysis or processing through the use of contrast agents provides for new types of care to be provided for specific anatomies and tissues. By way of example, the present techniques contemplate care in the areas of disease state detection, disease state diagnosis, establishment of care plans, treatment of disease states, organ and tissue transplants, follow-up of treatment and care, analysis of tissues and treatment, and image-guided surgery.

Moreover, the present techniques permit a level of care in these areas to be offered on tissues for which such analysis of the foregoing data types was simply unavailable in the past. These anatomies and tissues include tissues that do not offer a sufficient contrast in known imaging modalities, but for which the present technique offers enhanced acquisition and processing. Such anatomies and tissues include the liver, fat tissue, vasculature, tumors, stones, kidneys, and pancreas.

### DRAWINGS

These and other features, aspects, and advantages of the present invention will become better understood when the following detailed description is read with reference to the accompanying drawings in which like characters represent like parts throughout the drawings, wherein:

FIG. 1 is a diagrammatical overview of a contrast agent-based health care system employing imaging modalities for rendering patient care;

FIG. 2 is a diagrammatical overview of a contrast agent-based image-related workflow summarizing anatomies and tissues that may be imaged by specific types of data acquisition, and data types for specific types of health care and care purposes through the use of specific types of processing and analysis;

FIG. 3 is a flow diagram illustrating an exemplary workflow process for performing image data acquisition and analysis and providing health care based upon the systems summarized in FIGS. 1 and 2 in accordance with aspects of the present technique;

FIG. 4 is diagrammatical overview of certain key functional components and modules for performing the process set forth in FIG. 3;

FIG. 5 is a summary of a matrix of possible care paths contemplated by the present technique employing the various types of analysis and processing summarized in FIG. 2 on specific data types for specific health care purposes and specific anatomies or tissue;

FIG. 6 is an diagrammatical overview of an exemplary imaging system that may be employed in accordance with the present technique;

FIG. 7 is diagrammatical overview of an exemplary digital X-ray system that may be employed with the present technique;

FIG. 8 is an overview of an exemplary magnetic resonance imaging system that may be employed in accordance with aspects of the present technique;

FIG. 9 is a diagrammatical overview of an exemplary computed tomography imaging system that may be employed in the present technique;

FIG. 10 is an exemplary positron emission tomography imaging system that may be employed in accordance with aspects of the present technique;

FIG. 11 is a flow chart illustrating exemplary steps in performing contrast agent-based imaging and analysis of disease states in the liver as an example of the type of health care offered by the present technique and workflow summarized in the previous figures;

FIG. 12 is a diagram illustrating the acquisition of image data in a computed tomography system at multiple phases or stages in the progression of a contrast agent through a patient for implementation of the steps set forth in FIG. 11;

FIG. 13 is an exemplary image made based upon computed tomography information taken at a first time as indicated in FIG. 12;

FIG. 14 is an exemplary image made on a computed tomography system taken at a second time as indicated in FIG. 12, illustrating different tissues from those visible in FIG. 13 due to the progression of contrast agent;

FIG. 15 is an exemplary consolidated or fused image incorporating data from the image of FIG. 13 and the image of FIG. 14 for the purposes of visualization, localization, analysis and treatment of liver disease states as an example of the type of care offered by the present techniques; and

FIG. 16 is a diagrammatical representation of an image guided surgery installation incorporating aspects of the present techniques.

### DETAILED DESCRIPTION

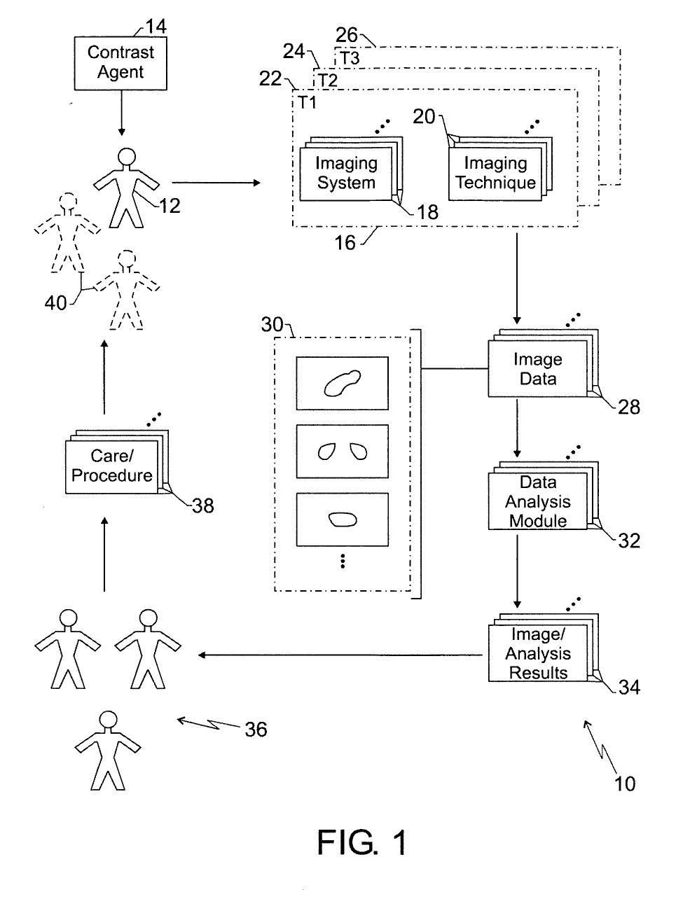

Turning now to the drawings, and referring first to FIG. 1, a contrast agent-based health care system 10 is illustrated diagrammatically. The system is based upon use of one or more imaging technologies that are used to collect data relating to internal tissues, organs, structures, and so forth in a patient 12. In accordance with the technique, the patient 12 is administered a contrast agent 14. The contrast agent may be administered in any one of range of conventional manners, such as orally or intravenously. As the contrast agent progresses through the target tissues of the patient, the patient is subjected to imaging procedures. Accordingly, an imaging component 16 is employed to collect data for later analysis and, where appropriate, image reconstruction.

The imaging component 16 will typically include one or more imaging systems 18 used in conjunction with one or more imaging techniques 20. As described more fully below, the imaging systems may include a range of imaging modalities, including digital X-ray systems, computed tomography systems, magnetic resonance imaging systems, positron emission tomography systems, ultrasound systems, X-ray tomosynthesis systems, and so forth. As will be appreciated by those skilled in the art, such systems are often considered to be different imaging “modalities” by virtue of their use of different imaging physics. The imaging techniques 20 may be considered different techniques that may be used on a single type of imaging system or modality system. Such techniques may include particular types of image data acquisition, specific types of data processing, various types of patient positioning and patient control, and so forth. By way of example only, within the X-ray field, imaging techniques may include various patient positioning and orientation to create projections that best show anatomies of interest. In the computed tomography imaging arena, various types of scans may be performed as imaging techniques. Such scans may include helical scans wherein a table is displaced in a scanner, various types of volumetric scanning, scout-mode scanning, as well as techniques for identifying various data windows of interest for image analysis and reconstruction. In the magnetic resonance imaging field, such techniques may include various pulse sequence descriptions that are specifically designed to create magnetic resonance echoes from various types of tissues, fluids, contrast agents, and the like. Such pulse sequence descriptions may also be designed to differentiate specific types of tissue or to best illustrate the progression of fluids or contrast agents within and between tissues.

As illustrated in FIG. 1, the imaging component 16, including the imaging systems 18 and imaging techniques 20 may be employed at different times, as indicated by blocks 22, 24 and 26. The provision of images at different times facilitates the comparison of similar tissues both within a specific patient and between patients or a population of patients. By way of example, imaging at different times may illustrate the progression of a disease state (e.g., the growth of a tumor), or trace the response of a disease state to a particular treatment. Moreover, it should be noted that these times may be remote from one another, such as removed from one another by days, weeks, months or even years. In other contexts, however, the times will be very close in proximity, such as for acquiring image data and processing the data during a procedure. In a present example described below, for example, such times are quite close in proximity in order to trace the progression of a contrast agent administered during a single examination sequence so as to permit imaging and analysis of different tissues that are affected by the contrast agent at different points in time as it progresses. Other procedures where the times may be quite close to one another include surgical procedures and interventions, such as minimally-invasive procedures used to treat vascular conditions, ablate tissues, place implants, aide in transplant, and so forth. As described more fully below, the times 22, 24 and 26 may also serve to collect image data on different patients to facilitate both the evaluation of health conditions in one or multiple patients, or to facilitate treatment of one patient, such as for sizing of soft tissues prior to a transplant.

The imaging component 16 will generate image data that is stored for immediate or later processing, as indicated at reference numeral 28 in FIG. 1. The image data may be stored in accordance with conventional techniques, such as in memory circuits of the imaging system itself, or in departmental or hospital storage systems, archive systems and so forth. The image data will typically include data encoding picture elements (pixels) or volume elements (voxels) either in a processed form, a raw form or a semi-processed form. In all of these cases, however, the image data will include data that can be analyzed for evaluation and, in most cases, eventual reconstruction of an image of target anatomies, as indicated generally by reference numeral 30 in FIG. 1. As noted above, in accordance with the present techniques, these anatomies may include the liver, fat tissues, vasculature, tumors, stones, kidneys and the pancreas.

Reference numeral 32 indicates one or more data analysis modules. These data analysis modules may be implemented at the imaging system itself, or within an institution, or completely removed from the institution. Indeed, the data analysis modules may be considered to include, depending upon the needs and type of data one or more appropriately programmed general purpose or application-specific computer with firmware or software designed to filter and process the image data. The functions of the data analysis modules are discussed in greater detail below. In general, however, the data analysis modules permit the raw or processed data from the imaging system to be analyzed for identification of tissues, differentiation of tissues, and further processing to segment, identify, diagnose, and to perform further functions on the image data or rendering of health care relating to the specific tissues imaged via the contrast agent. The data analysis modules may be of different types, depending upon the data type, the analysis to be performed, and the imaging system or even the imaging technique used to generate the image data. Ultimately, the data analysis modules provide image and analysis results which can be summarized or reconstructed, as indicated at reference numeral 34. These results and analyses may be rendered immediately, that is, during or immediately subsequent to the image data acquisition, such as for specific on-going procedures. In other cases, the image data and analysis results may be provided subsequently, such as for diagnosis and planning of treatment, or for following up on treatment. In certain cases the analysis results may be provided in forms other than image-based forms, including reports, textual summaries, and the like. In many situations, the results may be separately stored for remote transmission, printing, archiving, and so forth.

Ultimately, the results of the data analysis performed on the image data based upon the use of the contrast agent will be rendered to medical professionals, as indicated at reference numeral 36 in FIG. 1. These may include radiologists, specialized physicians, primary physicians, clinicians, and other health care professionals. As noted, the information may be provided both locally and immediately, such as during a procedure, or may be provided remotely and at subsequent times. In general, however, the information is provided for the purpose of permitting health care and specific procedure types as indicated by reference numeral 38 in FIG. 1. As described in greater detail below, in present embodiments, the procedures include specific procedures for the specific types of tissues images via the contrast agent. As noted above, where appropriate, the components illustrated in FIG. 1 may be employed for a population of patients, as indicated by reference numeral 40 in FIG. 1. These patients may include reference cases, such as from a general population or population known to exhibit specific characteristics or conditions. In other situations, the population will include a limited number of patients, such as organ donors.

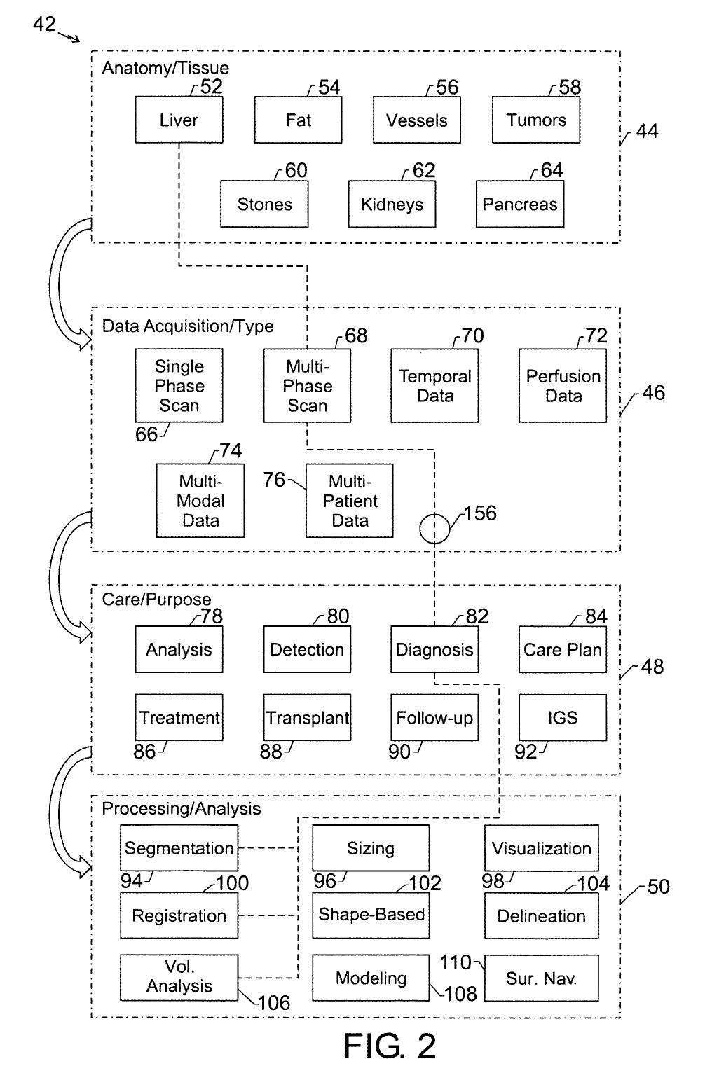

The system of FIG. 1 is designed to provide contrast agent-based, image-related health care for specific types of anatomies and tissues for using specific processing and analysis techniques and data types for specific care purposes. FIG. 2 summarizes the image-related workflow, indicated by reference numeral 42 available through the system of FIG. 1. In general, the workflow may be inclusive of various “paths” that were heretofore unavailable. Enablers for the paths include faster acquisition techniques than were heretofore available, particularly in computer tomography and magnetic resonance imaging. The techniques may also be enabled by the use of isotropic volume imaging and analysis. In such techniques, the dimensions of volumes considered (typically as voxels) are similar in dimensions in three orthogonal directions (i.e., X, Y, Z). A further enabler for the paths is the enhanced computational speed now available in many systems that allows for treatment of vast volumes of image data both within the imaging system itself, as well as subsequently on stored data. Again, however, the paths denoted in FIG. 2, and discussed in detail below, are believed to be heretofore unavailable and thus are new in the present context.

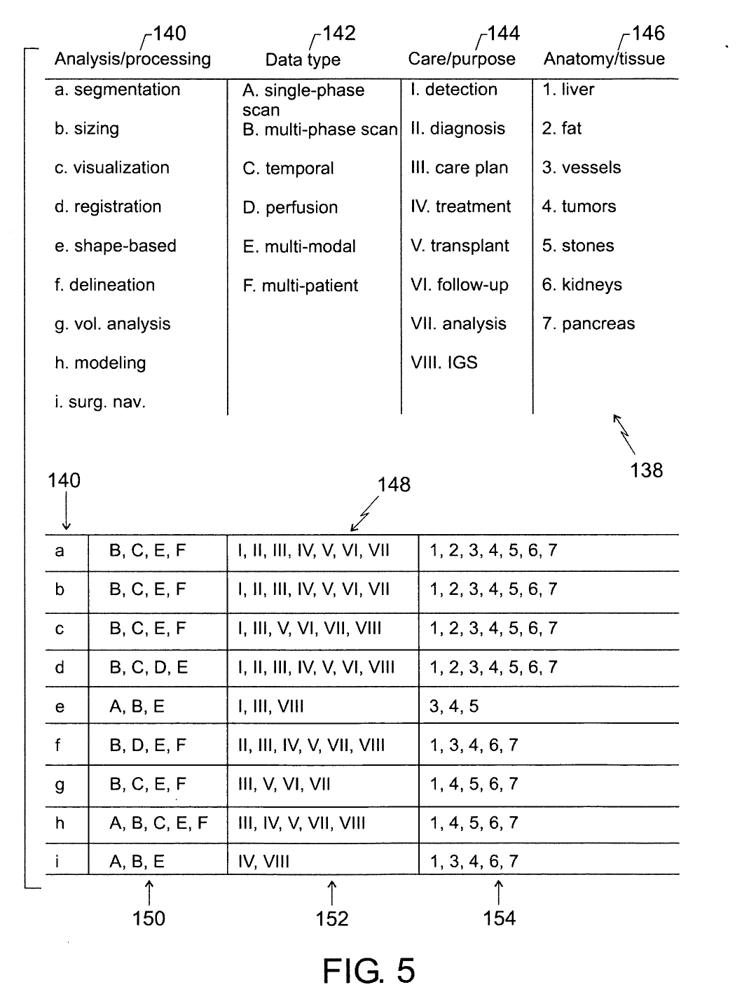

Applicants note that, as summarized herein, the present techniques permit new procedures and workflow paths to be established for treatment of anatomies and tissues based upon the administration of contrast agents. The paths include four components. As summarized in FIG. 2, these include the anatomies or tissue 44, the data acquisition or data type 46, the type of care or purpose for the imaging and analysis 48, and the particular processing or analysis performed on the data 50. Certain of the paths through each of the particular subcomponents illustrated in FIG. 2 are believed to be independently and separately new and unobvious, as discussed below with respect to FIG. 5, and the inventors are unaware of any proposed or present use of such paths in the art. In the event that, upon further review and examination, specific paths are found to be pre-existing in the field, however, the present techniques and the scope of claims made based on this disclosure are nevertheless intended to extend to other paths defined by intersection of one or more of the subcomponents in each component summarized in FIG. 2 to the extent that they are individually novel and unobvious.

In general, the tissues enumerated in the anatomy or tissue component 44 of FIG. 2 include specific tissues that can be imaged or differentiated through the use of a contrast agent with specific data acquisition types. Again, such imaging and analysis could not be performed in heretofore known systems on these types of tissues.

The specific anatomies or tissues for which imaging, analysis and care are contemplated in the present workflow processing are summarized as component 44 in FIG. 2. As enumerated in the figure, these include the liver 52, fat tissue 54, vessels and vasculature 56, tumors 58, stones 60, kidneys 62, and the pancreas 64. As will be appreciated by those skilled in art, while to some limited degree imaging of these tissues have been performed in the past, the present contrast agent-based processing permits greatly enhanced acquisition of image data on such tissues, followed by specific processing and analysis for specific care purposes.

The particular data acquisition techniques and data types, summarized as component 46 in FIG. 2 include single phase scans 66 and multi-phase scans 68. As used herein, these terms refer to phases in the progression of contrast agents through, within and between tissues. For example, in a specific case discussed below, different types of tissue may be affected by and therefore may be imaged and analyzed by a contrast agent as it progresses through the liver. Such use of the phases in the progression of the contrast agent may therefore allow for imaging and analysis of different types of tissues as they are differentially affected at different times by the contrast agent.

As also summarized in FIG. 2, the data may include temporal data 70. As used herein, the term temporal data refers to data acquired at different points in time. The data may be acquired with the same or different contrast agent, and at generally the same phase of progression of the contrast agent through, within or between the same tissues. The use of such temporal data, spaced in time by seconds, minutes, hours, days, weeks, or even more, permits the analysis of the increase or decrease in size, change in shape, change in texture, or other characteristics of a particular anatomy, such as tumors. These as such temporal data may be used to analyze the progression of a disease state or condition, as well as the response of a disease state or condition to treatment.

A further type of data contemplated by the present technique is profusion data as indicated by reference 72 in FIG. 2. As will be appreciated by those skilled in the art, profusion data may be thought of as four-dimensional data comprised of multiple three-dimensional image data sets over time. Such data may be used to image the flow of contrast agent, such as to characterize the uptake variability of the contract agent which may, itself, be characteristic of the presence of tumors or tumor growth.

Multi-modal data 74 may also be processed in accordance with the present techniques. As will be appreciated by those skilled in the art, this type of data will include data from different imaging modalities which may be used to compliment one another. Multi-modality data may include, for example, computed tomography image data and magnetic resonance image data.

Finally, a further type of data that is contemplated by the present technique is multi-patient data as indicated at reference numeral 76. Such data may be considered similar to temporal data 70, but for different patients. Such data may be used, for example, to analyze differences or distinctions between normal and diseased tissue, for sizing tissues and organs, such as for transplants, and so forth.

Based upon the particular anatomy or tissue to which care is to be provided, and upon the data acquisition or data type employed, each care path further includes a specific type of care to be provided or purpose for the imaging. That is, the analysis or processing algorithm employed may be adapted for the particular purpose or case goal. As discussed below, it is assumed herein that the basic techniques of the algorithms may be generally known, and that the algorithms may be adapted for the tissue, data type and care purpose without undue experimentation.

These are summarized in FIG. 2 as the care-purpose component 48. In accordance with the present technique, these paths may thus include analysis 78, detection 80, diagnosis 82, the establishment of a care plan 84, treatment 86, transplant 88, follow-up of a treatment 90 or image guided surgery 92. The analysis detection and diagnosis functions of the care may be considered generally similar, although the ultimate purpose may be somewhat distinct. For example, analysis tissue may be carried out in a general fashion to determine the nature of tissues, there position, size, and so forth. Detection may include such analysis, but may be directed toward detecting a specific condition in the tissues of a patient. Diagnosis, furthermore, typically includes analysis and detection followed by comparison of potentially diseased tissues with normal tissue structures to determine whether a particular disease state exists, and to characterize or classify the disease state.

Similarly, the establishment of a care plan as indicated at reference numeral 84, followed by treatment transplant and follow-up may be thought of as similar, although each of these purposes is, in fact, distinct. The establishment of a care plan 84, for example, may include treatment or may be provided simply for the purpose of informing patients of certain medical conditions that may affect or should affect their lifestyle, eating habits, and so forth. A care plan may be more comprehensive than specific treatments. Treatments 86 may, of course, include prescription of specific drugs, or may involve surgical interventions, ranging from non-invasive procedures to highly invasive surgeries, such as transplants as indicated specifically at reference numeral 88. As will be appreciated by those skilled in the art, such transplants are highly specialized procedures requiring extensive analysis of organs and their placement, size and compatibility. The present techniques provide for such analysis in a manner heretofore unavailable. The follow-up procedures, as indicated by reference numeral 90, may include analysis of the progression or resolution of certain conditions based upon a care plan, treatment, transplant or any other medical procedure. Moreover, such follow-up may simply be performed to monitor the progression or resolution of a medical condition.

Image guided surgery as indicated at reference numeral 92 in FIG. 2 may greatly benefit from the present techniques. In particular, various types of surgeries may be performed, particularly non-invasive surgeries or minimally invasive surgeries on tissues of the type indicated at component 44 based upon detailed imaging analysis provided by the use of contrast agents as described herein. The surgical procedure itself may be performed during an imaging sequence, or subsequent to the imaging sequence. In image guided surgery, the surgeon is informed of the location, shape, size, and any other critical parameters of the tissues to be targeted by the surgery, and may adapt the surgical procedures based upon the detailed imaging analysis. As described in one of the examples below, such surgical interventions may, for example, include ablation of tumors based upon high quality computed tomography images with fast low quality ultrasound imaging as a guide. Other types of image-based guides using contrast agents may, of course, be envisaged or developed based upon the present techniques.

Finally, several processing and analysis procedures are envisaged by the present technique as indicated by component 50 in FIG. 2. These include segmentation 94, sizing 96, visualization 98, registration 100, shape-based analysis 102, delineation 104 volumetric analysis 106, modeling 108 and surgical navigation 110. Each of these techniques will be discussed in greater detail below. However, as will be appreciated by those skilled in the art, such techniques, while developed for other tissues and anatomies, have heretofore unavailable for the types of anatomies and tissues listed in component 44 of FIG. 2 based upon the type of data listed in component 46 of FIG. 2 for the purposes of care listed in component 48 of FIG. 2.

It should also be noted that, based upon the components listed in FIG. 2, a wide range of data flow or workflow paths may be defined. By way of example, as indicated by the dashed line in FIG. 2, one such path may include the liver as a target tissue imaged through the use of multi-phase scanning. The multi-phase scanning of the liver may be performed, for example, for the purpose of diagnosis of tumors. The diagnosis may be performed through analysis of the multi-phase scanned data of the liver through one or more of the processing and analysis techniques listed, such as segmentation, registration and volumetric analysis. The particular example of the path illustrated by the dashed line in FIG. 2 is provided as a detailed exampled below. Again, it is believed that none of the paths defined by the components and sub-components of FIG. 2 in combinations summarized in FIG. 5 is in use or has been proposed in art. However, the present invention is considered to extend to any one of these paths or to only the new paths definable by the components and sub-components, where such use or development has taken place in the art.

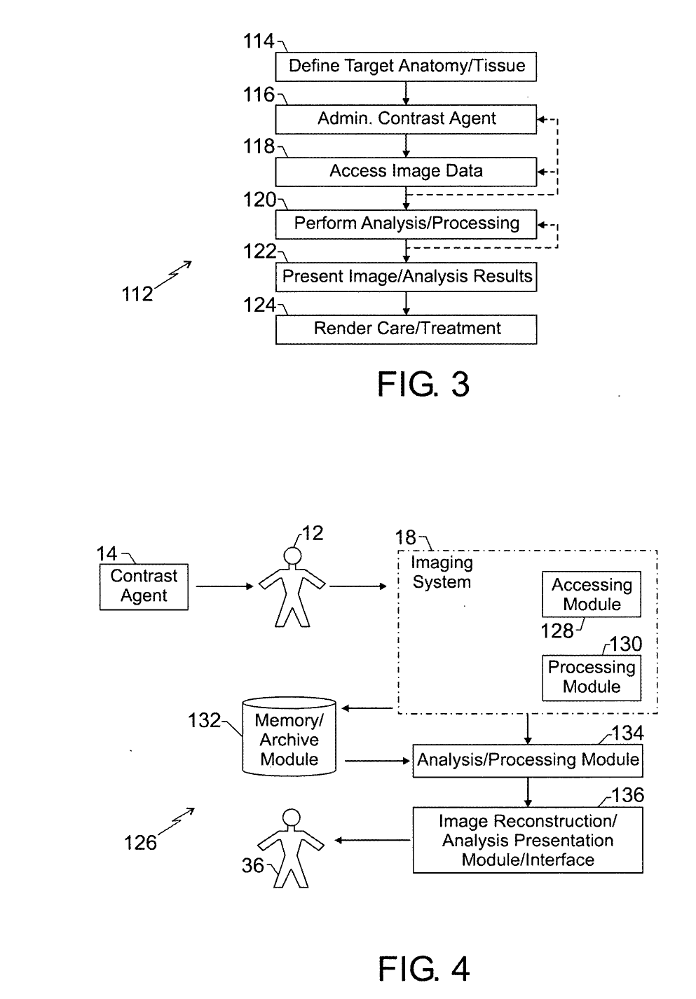

FIG. 3 illustrates exemplary steps in a workflow process 112 for performing the image data processing and analysis based upon the components summarized in FIG. 4. In general, the processing begins at a step 114 where a target anatomy or tissue is defined. Multiple such target anatomies or tissues may, of course, be defined, although in a typical example the contrast agent will be defined, selected, and administered based upon a single organ or type of tissue. At step 116 the contrast agent is administered and allowed to progress to, through, within and between target tissues. During this progression image data is acquired as indicated at step 118. As noted above, the image data may be acquired on any suitable imaging system, with any suitable technique being employed on the system. As indicated by the broken line in FIG. 3, image data may be acquired at multiple times, with the potential for multiple contrast agents being administered. Such will be the case, for example, where different modalities are employed, where image data is to be considered on a temporal basis or where comparisons are to be made between a specific patient and other patients. At step 120 the desired analysis and processing is performed.

As is also indicated by the broken line in FIG. 3, such analysis and processing may be performed multiple times or multiple types of analysis and processing may be performed. Moreover, such analysis and processing may be performed in parallel, where appropriate, or the results of one type of analysis and processing may be used as an input to subsequent analysis and processing. For example, the specific tissues may be segmented prior to registration with on another or comparative volumetric analysis.

At step 122, images may be reconstructed, where appropriate, or results of analysis may be otherwise presented. The presentation of the analysis may include reconstruction of images, or analyses may be presented in terms of text, numerical results, and so forth. At step 124 the desired care or treatment is performed based upon the reconstructed images and/or analysis results.

The principle components in a system for carrying out the workflow of FIG. 3 are illustrated in FIG. 4. In the specific example illustrated, the contrast agent 14 is administered to the patient 12, and the patient 12 is subjected to an imaging scan or session in the imaging system 18. As noted above, this may include subjecting the patient to multiple different contrast agents, such as different times, as well to imaging sequences on different types of systems or with different system settings. As discussed in greater detail below, the imaging system 18 will typically include an acquisition module 128 which is typically comprised of circuitry adapted to detect signals representative of tissues and materials at specific locations within the patient. Processing module 130 in the imaging system permits initial processing, such as filtration, analog-to-digital conversion, dynamic range adjustment, and any other suitable initial processing to be performed. Certain processing modules 130 within certain imaging systems may perform more detailed image data processing, and even image enhancement and reconstruction. Thus, the processing module 130, in conjunction with the acquisition module 128 may reconstruct images for display in real-time or near real-time, such as for image guided surgery.

In the example illustrated in FIG. 4, the image data is stored in a memory or archive module 132. In the simplest case, the memory may be included in the imaging system itself. In most cases of medical imaging, however, detailed image data, either raw, processed or semi-processed is stored in an archive, such as a picture archiving and communication system (PACS) for later use. Such is particularly the case where temporal data is to be used, or where the data is intended to be used in comparing patients or patient anatomies.

An analysis or processing module 134 permits the particular processing desired on the specific type of data included in the workflow path. As will be appreciated by those skilled in the art, such modules will most often include programming instructions for a general purpose or application-specific computer. The analysis and processing may be subject to operator intervention and definition, such as to specify dynamic ranges, windowing, to select specific fields of view, areas of interest, regions of interest, anatomies of interest, and so forth. Based upon the results provided by the analysis and processing module 134, an image reconstruction and analysis presentation module or interface 136 provides feedback to the care provider 36. The module 136 will typically vary with the imaging system type, although certain standardized modules or interfaces may be available. For example, the module or interface 136 may be associated directly with the imaging system, and adapted perform image reconstruction and presentation on the specific type of image data produced by the system. In other settings, the module or interface may be adapted to perform image reconstruction analysis of data from a range of different modalities, such as in a PACS workstation.

FIG. 5 illustrates a matrix of potential paths definable and contemplated by the present techniques. The paths include anatomies and tissues of the component 44 illustrated in FIG. 2, through data acquisition and data types illustrated in component 46, for care purpose illustrated in component 48 and through the use and specific processing and analysis techniques indicated at component 50 in FIG. 2. The sub-components may form a series of non-mutually exclusive listings as indicated by reference numeral 38 in FIG. 5. These sub-components are enumerated in columns 140, 142, 144 and 146. However, not all of the paths possible by the listings are presently contemplated. Rather, specific paths adapted for specific types of analysis and processing of specific types of data for specific care purposes and specific anatomies or tissues are contemplated that form a workflow matrix as indicated at reference numeral 148.

The matrix 148 is constructed based upon the particular analysis and processing attribute listed in column 140. These are denoted by lowercase letters in FIG. 5. Each of these analysis and processing attributes is applicable and may form a workflow path with any one of the data types listed in column 150 of the matrix 148. Moreover, for each of the combinations of analysis and processing combined with each of the data types, a specific care purpose may be included in the workflow path as indicated by reference numeral 152 in the matrix 148. Finally, the analysis and processing techniques in conjunction with the data types and the care purpose are presently contemplated to be applicable with specific anatomies and tissues as indicated by reference numeral 154 in the matrix 148. It should be noted that not all of the possible workflow paths are thus contemplated. Rather, specific workflow paths are contemplated for the processing, data type, care purpose and anatomy.

Again, the present technique is specifically contemplated to include combinations of components from the listings provided herein. The summarized combinations are all believed to be new to the present technique and were unavailable through prior techniques. Certain of the combinations may be further developed and may prove to be of particular interest for users. Where specific combinations are known in the art or can be demonstrated to have pre-existed the present technique, these are specifically excluded from the protection sought by the inventors. However, the protection contemplated by the inventors is intended to extend to all of the combinations which are not demonstrated to be present in the prior art.

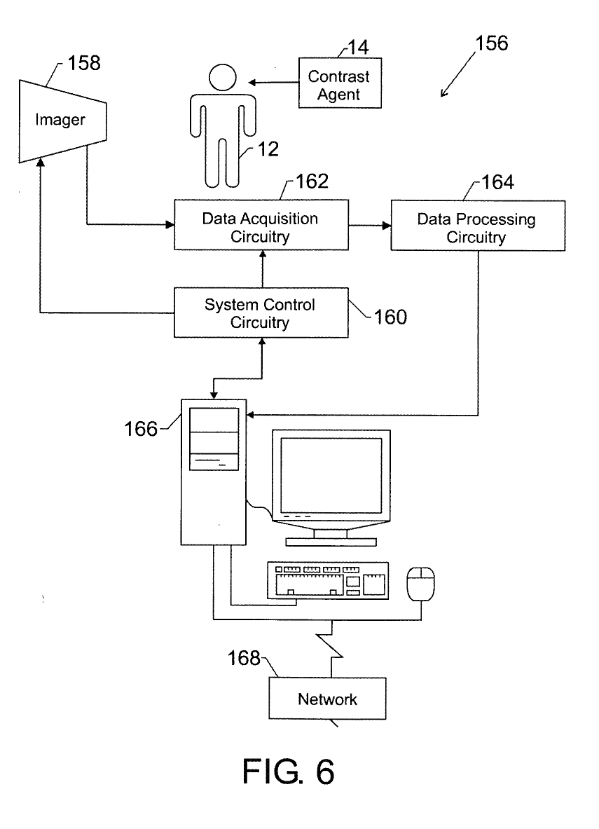

Imaging Systems and Techniques: Various imaging resources may be available for diagnosing medical events and conditions in both soft and hard tissue, and for analyzing structures and function of specific anatomies. Moreover, imaging systems are available which can be used during surgical interventions, such as to assist in guiding surgical components through areas which are difficult to access or impossible to visualize. FIG. 6 provides a general overview for exemplary imaging systems, and subsequent figures offer somewhat greater detail into the major system components of specific modality systems.

Referring to FIG. 6, an imaging system 156 generally includes some type of imager 158 which detects signals and converts the signals to useful data. As described more fully below, the imager 158 may operate in accordance with various physical principles for creating the image data. In general, however, image data indicative of regions of interest in a patient are created by the imager either in a conventional support, such as photographic film, or in a digital medium.

The imager operates under the control of system control circuitry 160. The system control circuitry may include a wide range of circuits, such as radiation source control circuits, timing circuits, circuits for coordinating data acquisition in conjunction with patient or table of movements, circuits for controlling the position of radiation or other sources and of detectors, and so forth. The imager 158, following acquisition of the image data or signals, may process the signals, such as for conversion to digital values, and forwards the image data to data acquisition circuitry 162. In the case of analog media, such as photographic film, the data acquisition system may generally include supports for the film, as well as equipment for developing the film and producing hard copies that may be subsequently digitized. For digital systems, the data acquisition circuitry 162 may perform a wide range of initial processing functions, such as adjustment of digital dynamic ranges, smoothing or sharpening of data, as well as compiling of data streams and files, where desired. The data is then transferred to data processing circuitry 164 where additional processing and analysis are performed. For conventional media such as photographic film, the data processing system may apply textual information to films, as well as attach certain notes or patient-identifying information. For the various digital imaging systems available, the data processing circuitry perform substantial analyses of data, ordering of data, sharpening, smoothing, feature recognition, and so forth.

Ultimately, the image data is forwarded to some type of operator interface 166 for viewing and analysis. While operations may be performed on the image data prior to viewing, the operator interface 166 is at some point useful for viewing reconstructed images based upon the image data collected. It should be noted that in the case of photographic film, images are typically posted on light boxes or similar displays to permit radiologists and attending physicians to more easily read and annotate image sequences. The images may also be stored in short or long term storage devices, for the present purposes generally considered to be included within the interface 166, such as picture archiving communication systems. The image data can also be transferred to remote locations, such as via a network 168. It should also be noted that, from a general standpoint, the operator interface 166 affords control of the imaging system, typically through interface with the system control circuitry 160. Moreover, it should also be noted that more than a single operator interface 136 may be provided. Accordingly, an imaging scanner or station may include an interface which permits regulation of the parameters involved in the image data acquisition procedure, whereas a different operator interface may be provided for manipulating, enhancing, and viewing resulting reconstructed images.

The following is a more detailed discussion of specific imaging modalities based upon the overall system architecture outlined in FIG. 6.

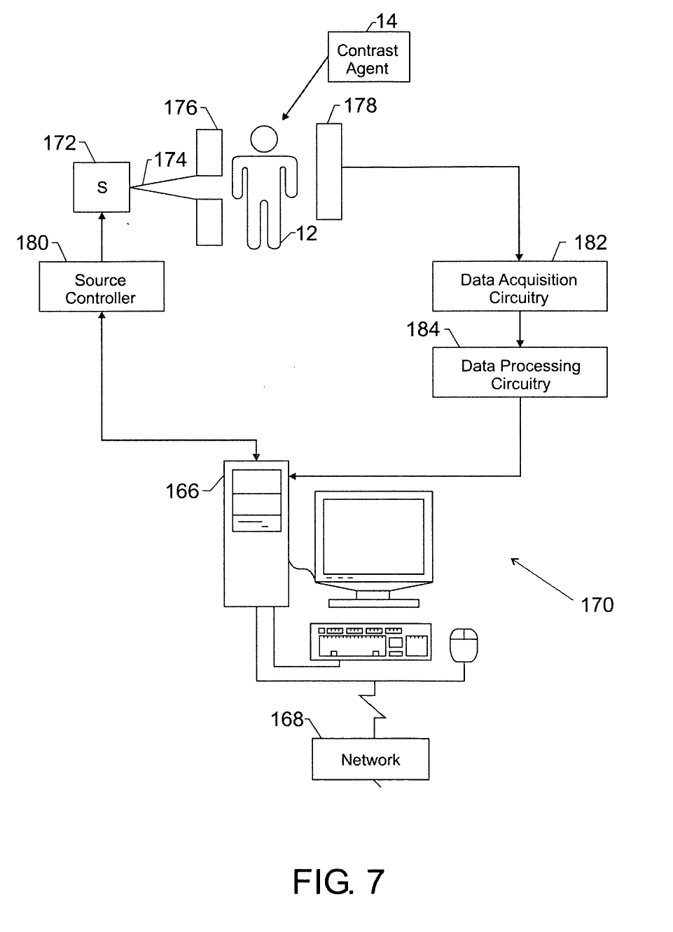

X-ray: FIG. 7 generally represents a digital X-ray system 170. It should be noted that, while reference is made in FIG. 7 to a digital system, conventional X-ray systems may, of course, be provided as controllable and prescribable resources in the present technique. In particular, conventional X-ray systems may offer extremely useful tools both in the form of photographic film, and digitized image data extracted from photographic film, such as through the use of a digitizer.

System 170 illustrated in FIG. 7 includes a radiation source 172, typically an X-ray tube, designed to emit a beam 174 of radiation. The radiation may be conditioned or adjusted, typically by adjustment of parameters of the source 172, such as the type of target, the input power level, and the filter type. The resulting radiation beam 174 is typically directed through a collimator 176 which determines the extent and shape of the beam directed toward patient 12. A portion of the patient 4 is placed in the path of beam 174, and the beam impacts a digital detector 178.

Detector 178, which typically includes a matrix of pixels, encodes intensities of radiation impacting various locations in the matrix. A scintillator converts the high energy X-ray radiation to lower energy photons which are detected by photodiodes within the detector. The X-ray radiation is attenuated by tissues within the patient, such that the pixels identify various levels of attenuation resulting in various intensity levels which will form the basis for an ultimate reconstructed image.

Control circuitry and data acquisition circuitry are provided for regulating the image acquisition process and for detecting and processing the resulting signals. In particular, in the illustration of FIG. 7, a source controller 180 is provided for regulating operation of the radiation source 172. Other control circuitry may, of course, be provided for controllable aspects of the system, such as a table position, radiation source position, and so forth. Data acquisition circuitry 182 is coupled to the detector 178 and permits readout of the charge on the photodetectors following an exposure. In general, charge on the photodetectors is depleted by the impacting radiation, and the photodetectors are recharged sequentially to measure the depletion. The readout circuitry may include circuitry for systematically reading rows and columns of the photodetectors corresponding to the pixel locations of the image matrix. The resulting signals are then digitized by the data acquisition circuitry 182 and forwarded to data processing circuitry 184.

The data processing circuitry 184 may perform a range of operations, including adjustment for offsets, gains, and the like in the digital data, as well as various imaging enhancement functions. The resulting data is then forwarded to an operator interface or storage device for short or long-term storage. The images reconstructed based upon the data may be displayed on the operator interface, or may be forwarded to other locations, such as via a network 168 for viewing. Also, digital data may be used as the basis for exposure and printing of reconstructed images on a conventional hard copy medium such as photographic film.

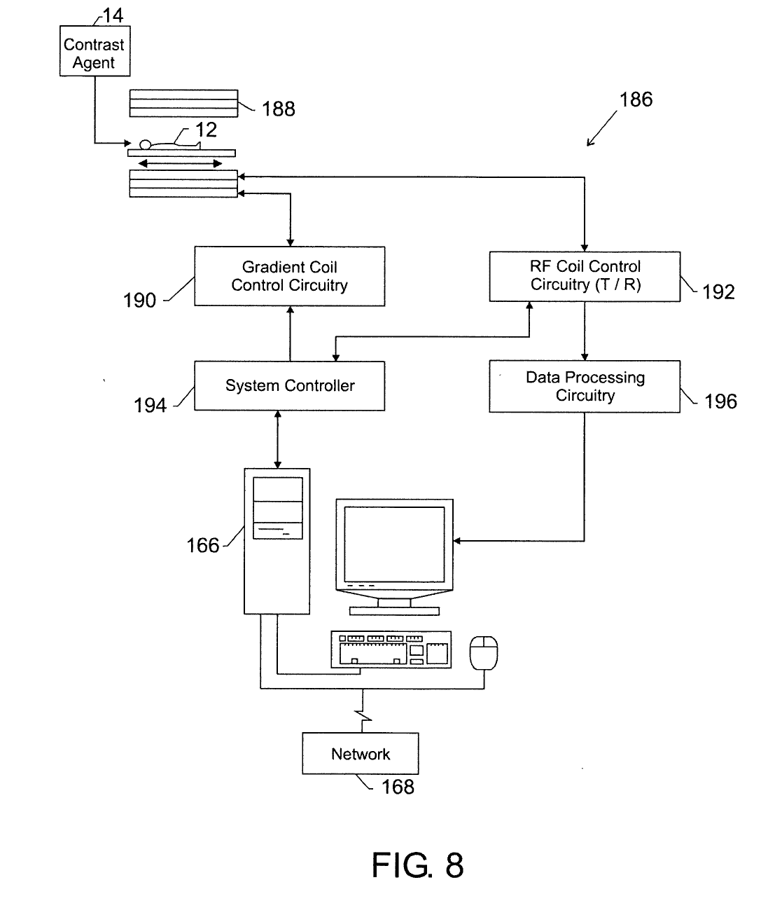

MR: FIG. 8 represents a general diagrammatical representation of a magnetic resonance imaging system 186. The system includes a scanner 188 in which a patient is positioned for acquisition of image data. The scanner 188 generally includes a primary magnet for generating a magnetic field which influences gyromagnetic materials within the patient's body. As the gyromagnetic material, typically water and metabolites, attempts to align with the magnetic field, gradient coils produce additional magnetic fields which are orthogonally oriented with respect to one another. The gradient fields effectively select a slice of tissue through the patient for imaging, and encode the gyromagnetic materials within the slice in accordance with phase and frequency of their rotation. A radio-frequency (RF) coil in the scanner generates high frequency pulses to excite the gyromagnetic material and, as the material attempts to realign itself with the magnetic fields, magnetic resonance signals are emitted which are collected by the radio-frequency coil.

The scanner 188 is coupled to gradient coil control circuitry 190 and to RF coil control circuitry 192. The gradient coil control circuitry permits regulation of various pulse sequences which define imaging or examination methodologies used to generate the image data. Pulse sequence descriptions implemented via the gradient coil control circuitry 190 are designed to image specific slices, anatomies, as well as to permit specific imaging of moving tissue, such as blood, and defusing materials. The pulse sequences may allow for imaging of multiple slices sequentially, such as for analysis of various organs or features, as well as for three-dimensional image reconstruction. The RF coil control circuitry 192 permits application of pulses to the RF excitation coil, and serves to receive and partially process the resulting detected MR signals. It should also be noted that a range of RF coil structures may be employed for specific anatomies and purposes. In addition, a single RF coil may be used for transmission of the RF pulses, with a different coil serving to receive the resulting signals.

The gradient and RF coil control circuitry function under the direction of a system controller 194. The system controller implements pulse sequence descriptions which define the image data acquisition process. The system controller will generally permit some amount of adaptation or configuration of the examination sequence by means of an operator interface 166.

Data processing circuitry 196 receives the detected MR signals and processes the signals to obtain data for reconstruction. In general, the data processing circuitry 196 digitizes the received signals, and performs a two-dimensional fast Fourier transform on the signals to decode specific locations in the selected slice from which the MR signals originated. The resulting information provides an indication of the intensity of MR signals originating at various locations or volume elements (voxels) in the slice. Each voxel may then be converted to a pixel intensity in image data for reconstruction. The data processing circuitry 196 may perform a wide range of other functions, such as for image enhancement, dynamic range adjustment, intensity adjustments, smoothing, sharpening, and so forth. The resulting processed image data is typically forwarded to an operator interface for viewing, as well as to short or long-term storage. As in the case of foregoing imaging systems, MR image data may be viewed locally at a scanner location, or may be transmitted to remote locations both within an institution and remote from an institution such as via a network connection 168.

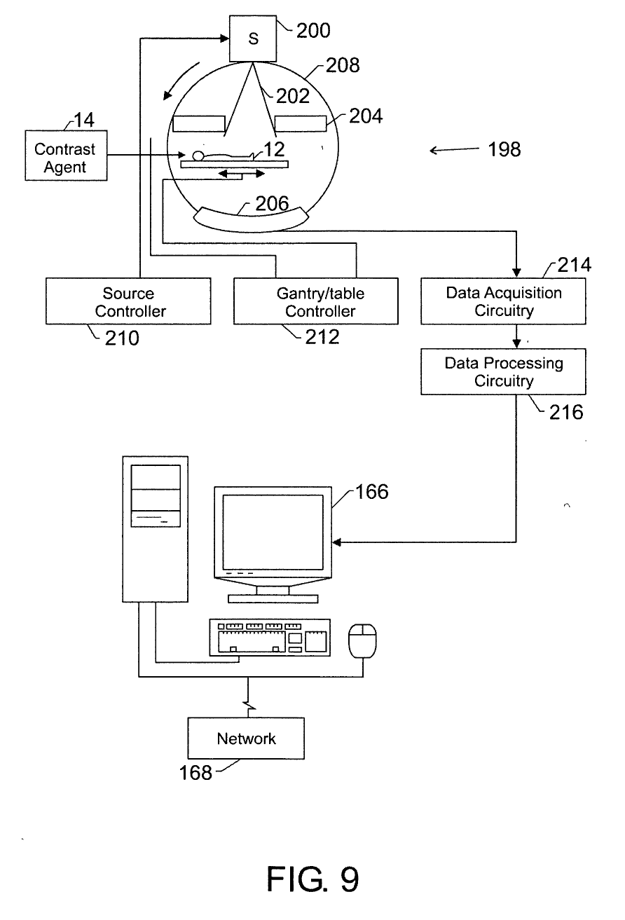

CT: FIG. 9 illustrates the basic components of a computed tomography (CT) imaging system. The CT imaging system 198 includes a radiation source 200 which is configured to generate X-ray radiation in a fan-shaped beam 202. A collimator 204 defines limits of the radiation beam. The radiation beam 202 is directed toward a curved detector 206 made up of an array of photodiodes and transistors which permit readout of charges of the diodes depleted by impact of the radiation from the source 200. The radiation source, the collimator and the detector are mounted on a rotating gantry 208 which enables them to be rapidly rotated (such as at speeds of two rotations per second).

During an examination sequence, as the source and detector are rotated, a series of view frames are generated at angularly-displaced locations around a patient 4 positioned within the gantry. A number of view frames (e.g. between 500 and 1000) are collected for each rotation, and a number of rotations may be made, such as in a helical pattern as the patient is slowly moved along the axial direction of the system. For each view frame, data is collected from individual pixel locations of the detector to generate a large volume of discrete data. A source controller 210 regulates operation of the radiation source 260, while a gantry/table controller 212 regulates rotation of the gantry and control of movement of the patient.

Data collected by the detector is digitized and forwarded to a data acquisition circuitry 214. The data acquisition circuitry may perform initial processing of the data, such as for generation of a data file. The data file may incorporate other useful information, such as relating to cardiac cycles, positions within the system at specific times, and so forth. Data processing circuitry 216 then receives the data and performs a wide range of data manipulation and computations.

In general, data from the CT scanner can be reconstructed in a range of manners. For example, view frames for a full 360° of rotation may be used to construct an image of a slice or slab through the patient. However, because some of the information is typically redundant (imaging the same anatomies on opposite sides of a patient), reduced data sets comprising information for view frames acquired over 180° plus the angle of the radiation fan may be constructed. Alternatively, multi-sector reconstructions are utilized in which the same number of view frames may be acquired from portions of multiple rotational cycles around the patient. Reconstruction of the data into useful images then includes computations of projections of radiation on the detector and identification of relative attenuations of the data by specific locations in the patient. The raw, the partially processed, and the fully processed data may be forwarded for post-processing, storage and image reconstruction. The data may be available immediately to an operator, such as at an operator interface 166, and may be transmitted remotely via a network connection 168.

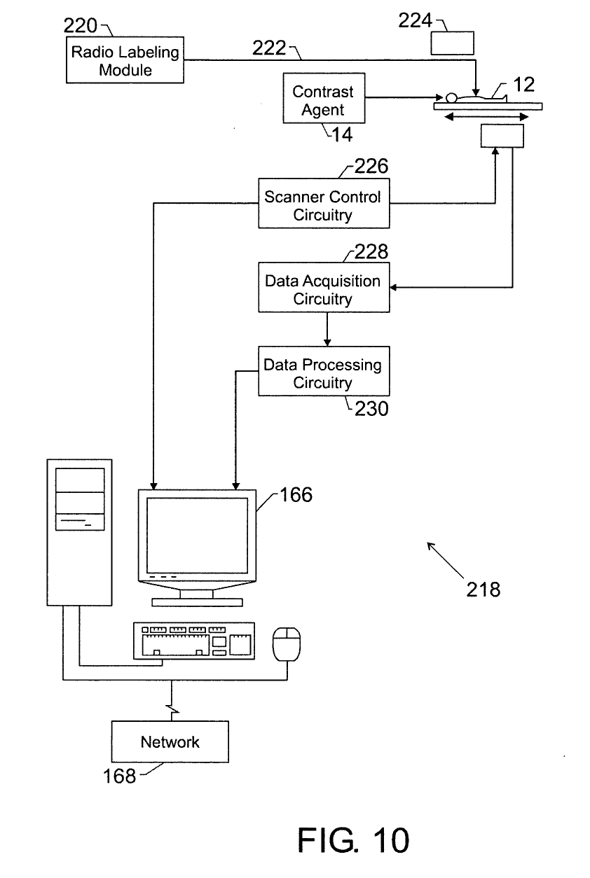

PET: FIG. 10 illustrates certain basic components of a positron emission tomography (PET) imaging system. The PET imaging system 218 includes a radio-labeling module 220 which is sometimes referred to as a cyclotron. The cyclotron is adapted to prepare certain tagged or radio-labeled materials, such as glucose, with a radioactive substance. The radioactive substance is then injected into a patient 12 as indicated at reference numeral 222. The patient is then placed in a PET scanner 224. The scanner detects emissions from the tagged substance as its radioactivity decays within the body of the patient. In particular, positrons, sometimes referred to as positive electrons, are emitted by the material as the radioactive nuclide level decays. The positrons travel short distances and eventually combine with electrons resulting in emission of a pair of gamma rays. Photomultiplier-scintillator detectors within the scanner detect the gamma rays and produce signals based upon the detected radiation.

The scanner 224 operates under the control of scanner control circuitry 226, itself regulated by an operator interface 166. In most PET scans, the entire body of the patient is scanned, and signals detected from the gamma radiation are forwarded to data acquisition circuitry 228. The particular intensity and location of the radiation can be identified by data processing circuitry 230, and reconstructed images may be formulated and viewed on operator interface 166, or the raw or processed data may be stored for later image enhancement, analysis, and viewing. The images, or image data, may also be transmitted to remote locations via a network link 168.

PET scans are typically used to detect cancers and to examine the effects of cancer therapy. The scans may also be used to determine blood flow, such as to the heart, and may be used to evaluate signs of coronary artery disease. Combined with a myocardial metabolism study, PET scans may be used to differentiate non-functioning heart muscle from heart muscle that would benefit from a procedure, such as angioplasty or coronary artery bypass surgery, to establish adequate blood flow. PET scans of the brain may also be used to evaluate patients with memory disorders of undetermined causes, to evaluate the potential for the presence of brain tumors, and to analyze potential causes for seizure disorders. In these various procedures, the PET image is generated based upon the differential uptake of the tagged materials by different types of tissue.

Fluorography: Fluoroscopic or fluorography systems consist of X-ray image intensifiers coupled to photographic and video cameras. In digital systems, the basic fluoroscopic system may be essentially similar to that described above with reference to FIG. 7. In simple systems, for example, an image intensifier with a video camera may display images on a video monitor, while more complex systems might include high resolution photographic cameras for producing still images and cameras of different resolutions for producing dynamic images. Digital detectors such as those used on digital X-ray systems are also used in such fluoroscopic systems. The collected data may be recorded for later reconstruction into a moving picture-type display. Such techniques are sometimes referred to as cine-fluorography. Such procedures are widely used in cardiac studies, such as to record movement of a living heart. Again, the studies may be performed for later reference, or may also be performed during an actual real-time surgical intervention.

As in conventional X-ray systems, the camera used for fluorography systems receives a video signal which is collected by a video monitor for immediate display. A video tape or disk recorder may be used for storage and later playback. The computer system or data processing circuitry may perform additional processing and analysis on the image data both in real-time and subsequently.

The various techniques used in fluorography systems may be referred to as video-fluoroscopy or screening, and digital fluorography. The latter technique is replacing many conventional photography-based methods and is sometimes referred to as digital spot imaging (DSI), digital cardiac imaging (DCI) and digital vascular imaging (DVI)/digital subtraction angiography (DSA), depending upon the particular clinical application. A hard-copy device, such as a laser imager, is used for to output hard copies of digital images. Moreover, fluoroscopic techniques may be used in conjunction with conventional X-ray techniques, particularly where a digital X-ray detector is employed as described above. That is, high-energy X-ray images may be taken at intervals interspersed with fluoroscopic images, the X-ray images providing a higher resolution or clarity in the images, while the fluoroscopic images provide real-time movement views.

Mammography: Mammography generally refers to specific types of imaging, commonly using low-dose X-ray systems and high-contrast, high-resolution film, or digital X-ray systems as described above, for examination of the breasts. Other mammography systems may employ CT imaging systems of the type described above, collecting sets of information which are used to reconstruct useful images. A typical mammography unit includes a source of X-ray radiation, such as a conventional X-ray tube, which may be adapted for various emission levels and filtration of radiation. An X-ray film or digital detector is placed in an oppose location from the radiation source, and the breast is compressed by plates disposed between these components to enhance the coverage and to aid in localizing features or abnormalities detectable in the reconstructed images. In general, the features of interest, which may include such anatomical features as microcalcifications, various bodies and lesions, and so forth, are visible in the collected data or on the exposed film due to differential absorption or attenuation of the X-ray radiation as compared to surrounding tissues. Mammography plays a central role in the early detection of cancers which can be more successfully treated when detected at very early stages.

Sonograph: Sonography imaging techniques generally include ultrasonography, employing high-frequency sound waves rather than ionizing or other types of radiation. The systems include a probe which is placed immediately adjacent to a patient's skin on which a gel is disposed to facilitate transmission of the sound waves and reception of reflections. Reflections of the sound beam from tissue planes and structures with differing acoustic properties are detected and processed. Brightness levels in the resulting data are indicative of the intensity of the reflected sound waves.

Ultrasonography is generally performed in real-time with a continuous display of the image on a video monitor. Freeze-frame images may be captured, such as to document views displayed during the real-time study. In ultrasound systems, as in conventional radiography systems, the appearance of structures is highly dependent upon their composition. For example, water-filled structures (such as a cyst) appear dark in the resulting reconstructed images, while fat-containing structures generally appear brighter. Calcifications, such as gallstones, appear bright and produce a characteristic shadowing artifact.

When interpreting ultrasound studies, radiologists and clinicians generally use the terminology “echogeneity” to describe the brightness of an object. A “hypoechoic” structure appears dark in the reconstructed image, while a “hyperechoic” structure appears bright.

Ultrasonography presents certain advantages over other imaging techniques, such as the absence of ionizing radiation, the high degree of portability of the systems, and their relatively low cost. In particular, ultrasound examinations can be performed at a bedside or in an emergency department by use of a mobile system. The systems are also excellent at distinguishing whether objects are solid or cystic. As with other imaging systems, results of ultrasonography may be viewed immediately, or may be stored for later viewing, transmission to remote locations, and analysis.

Infrared: Clinical thermography, otherwise known as infrared imaging, is based upon a careful analysis of skin surface temperatures as a reflection of normal or abnormal human physiology. The procedure is commonly performed either by the direct application of liquid crystal plates to a part of the body, or via ultra-sensitive infrared cameras through a sophisticated computer interface. Each procedure extrapolates the thermal data and forms an image which may be evaluated for signs of possible disease or injury. Differences in the surface temperature of the body may be indicative of abnormally enhanced blood flow, for example, resulting from injury or damage to underlying tissues.

Nuclear: Nuclear medicine involves the administration of small amounts of radioactive substances and the subsequent recording of radiation emitted from the patient at specific loci where the substances accumulate. There are a wide variety of diagnostic and therapeutic applications of nuclear medicine. In general, nuclear medicine is based upon the spontaneous emission of energy in the form of radiation from specific types of nuclei. The radiation typically takes the form of alpha beta and gamma rays. The nuclei are used in radiopharmaceuticals as tracers which can be detected for imaging, or whose radiation can serve for treatment purposes.

A tracer is a substance that emits radiation and can be identified when placed in the human body. Because the tracers can be absorbed differently by different tissues, their emissions, once sensed and appropriately located in the body, can be used to image organs, and various internal tissues. Radiopharmaceuticals are typically administered orally or intravenously, and tend to localize in specific organs or tissues. Scanning instruments detect the radiation produced by the radiopharmaceuticals and images can be reconstructed based upon the detected signals. Radioactive analysis of biologic specimens may also be performed by combining samples from the patient, such as blood or urine, with radioactive materials to measure various constituents of the samples.

In treatment, radioactive materials may be employed due to the emissions they produce in specific tissues in which they are absorbed. Radioactive iodine, for example, may be trapped within cancerous tissue without excessive radiation to surrounding healthy tissue. Such compounds are used in various types of treatment, such as for thyroid cancer. Because the iodine tends to pass directly to the thyroid, small doses of radioactive iodine are absorbed in the gland for treatment or diagnostic purposes. For diagnosis, a radiologist may determine whether too little or too much iodine is absorbed, providing an indication of hypothyroidism or hyperthyroidism, respectively.

Other types of imaging in nuclear medicine may involve the use of other compounds. Technetium, for example, is a radiopharmaceutical substance which is combined with a patient's white blood cells, and may be used to identify metastasis or spread of cancer in the bone. Following a period of settling, scans of specific limbs or of the entire body may be performed to identify whether metastasis can be diagnosed. Technetium may also be used to identify abnormalities in the liver or gallbladder, such as blockages due to gallstones. The substances also used in radionuclide ventriculograms. In such procedures, a sample of the patient's blood is removed (such as approximately 10 cm³) and radioactive technetium is chemically attached to the red blood cells. The blood is then injected back into the patient, and its circulation through the heart is traced and imaged.

Other uses for technetium in nuclear medicine include the diagnosis of appendicitis, due to the inflammation which occurs and the presence of white blood cells in the organ. Similarly, techniques involving technetium may be used for the diagnosis of abdominal inflammations and infections.

In radiation oncology known or possible extents tumors may be determined, and radiation employed to attack tumorous cells while avoiding major injury to surrounding healthy cells. External beam therapy, for example, involves radiation from a linear accelerator, betatron or cobalt machine that is targeted to destroy cancers at known locations. In brachytherapy, radioactive sources such as iodine, cesium or iridium are combined into or alongside a tumor. In another cancer therapy, known as boron neutron capture therapy (MNCT), alpha particles are produced by non-radioactive pharmaceuticals containing boron. Subsequent neutron beam irradiation causes neutrons to react with the boron in a tumor to generate alpha particles that aide in destroying the tumor.

Radioactive nuclides can be naturally-occurring or may be produced in reactors, cyclotrons, generators, and so forth. For radiation therapy, oncology, or other applications in nuclear medicine, radiopharmaceuticals are artificially produced. The radiopharmaceuticals have relatively short half-lives, such that they may be employed for their intended purpose, and degrade relatively rapidly to non-toxic substances.

Thermoacoustic: Thermoacoustic imaging systems are based upon application of short pulses of energy to specific tissues. The energy is created and applied to cause portions of the energy to be absorbed by a patient's tissue. Due to heating of the tissue, the tissue is caused to expand and an acoustic wave is thereby generated. Multi-dimensional image data can be obtained which is related to the energy absorption of the tissue. The energy may be applied in short pulses of radio-frequency (RF) waves. The resulting thermoacoustic emissions are then detected with an array of ultrasonic detectors (transducers).

Thermoacoustic scanners consist generally of an imaging tank, a multi-channel amplifier and an RF generator. The generator and the other components of the scanner are generally positioned in an RF-shielded room or environment. A digital acquisition system is provided along with a rotational motor for acquiring the thermoacoustic emission signals. A processing system then filters the signals, and processes them in digital form for image reconstruction. In general, the image contrast is determined by the energy delivered to the patient, and image spatial resolution is determined by the sound propagation properties and the detector geometry.

Image Processing and Analysis: As noted above, various types of image processing and analysis may be performed on the image data for the target anatomies and tissues. The types and combinations of processing and analysis performed will be selected based upon the purpose and care to be provided. Moreover, the processing and analysis summarized in the matrix of FIG. 5 is considered to be suitable for certain purposes, certain types of data, and certain anatomies. These may be summarized generally as follows.

Segmentation: Segmentation generally refers to the selection and identification of the bounds of specific types of tissues. In general, a region of interest (ROI) may be defined to calculate features in the image data. The ROI can be defined in several ways, such as using an entire data set or using only a part of the data. The candidate region may be selected, for example, automatically or by a user in a specific region of an image. In certain segmentation techniques, an initial starting point or a seed may be placed automatically or by the user, and the limits of a feature, such as a tumor, vessel, organ, or any other feature of interest may be identified by expanding a candidate boundary out or contracting a candidate boundary in until a limit is reached. Several techniques or their combinations may be used for this purpose. These may include, but are not limited to, iterative thresholding, k-means segmentation, edge detection, edge linking, curve fitting, curve smoothing, 2D/3D morphological filtering, region growing, fuzzy clustering, image/volume measurements, heuristics, knowledge-based rules, decision trees, and neural networks.

The segmentation of a region of interest can be performed manually and/or automatically. In manual segmentation, data is typically displayed for a user, and the user selects a region using an input device such as a mouse or other suitable interface (e.g., a touch screen, eye-tracking, voice commands, etc.). An automated segmentation algorithm may use prior knowledge, such as the shape and size of a mass, to automatically delineate the ROI. Semi-automated techniques may use a combination of the above two methods. In many such techniques, it may be advantageous to use the acquisition parameters of the object being imaged in the segmentation to improve the robustness of the procedure. Thus, the segmentation parameters may be specifically adapted to the image data type, the anatomy to be viewed, settings of the system, and so forth. In certain settings such information may be available either in the image examination record, or in the image itself, such as in a DICOM header.

Sizing: Sizing of specific anatomies may be performed in conjunction with segmentation or delineation discussed herein. In general, there are many reasons to actually determine the size of an anatomical object. These might include determination of an increase in the diameter of a vessel as an indication of an aneurysm, or the decrease in the same diameter as an indication of stenosis. In the case of tumors, an important characteristic to their malignancy is their doubling time that is calculated by comparing the volume of the tumor over time as in the case of follow-up or temporal exams.

Accurate size measurements are key to many diagnostic decisions. For example, images of objects are not completely accurate as noise, system transfer functions and object motion contribute to the fuzziness of their boundaries. A typical imaging system can be modeled as a series of additive transfer functions that include photonic noise, an imaging transfer function, an effective transfer function for a partial volume, with the convolution of these being the effective overall system operation. Accurate sizing thus involves accurate segmentation plus the compensation for noise and the system transfer function. In the present context, sizing is intended to relate to point linear, area, and volume measurement. Such measurements may be performed based upon the identification of the limits of a particular anatomy, followed by measurement of the anatomy with knowledge of the scaling in the image data (i.e., the pixel or voxel resolution).

Visualization: Visualization in the present context may be performed in two or three dimensions, or indeed in four dimensions on time-differentiated data. In general, the term volume rendering may be used to described visualization in three dimensions. Such techniques are based upon sampled functions of three spatial dimensions and typically include computing two 2D projections of a semi-transparent volume.

For volume rendering, stacks of two-dimensional parallel plane images may be employed. The particular image data acquisition system may provide such stacks, or the stacks may be determined by processing, such as in the case of X-ray tomosynthesis. The visualization technique provides for viewing the resulting volume in one or many view points.

Available method for visualization include rendering of voxels in binary partitioned space, marching cubes, ray casting, ray tracing, and texture mapping, to mention but a few. By rendering in binary partitioned space, the choices are made for each voxel for visualization purposes and placement. The technique of marching cubes solves problems with binary partitioned space processing, such as the production of “blocky” image. Ray casting technique map the image plane into data, and for each pixel in a final image, shoots rays from the pixel into the volume data and intersects the ray with each data point until the ray exits the volume or the opacity accumulates enough density to become opaque. Input values may not fall exactly on a ray, however. The technique does, however, solve important limitations in surface extraction techniques, namely the manner in which a projection is displayed of a thin shell in the acquisition space. Ray tracing involves mapping of volume data directly onto the image plane. For each voxel, the point is mapped onto the image plane and its contribution is added to the accumulating image. Texture mapping involves the adding of visual richness into a rendered image. Such techniques may involve bilinear or trilinear interpolation to sample data relating to such textures.

Registration: Where regions or volumes of interest are to be compared, contrasted, or otherwise fit to or with one another, registration techniques provide for identifying how this is to be done, and how the features are to be oriented and placed with respect to one another. Such techniques may be performed for a specific of interest, or for an entire region of an image or an entire image. Where the regions or volumes of interest for registration are small, rigid body registration transformations including translation, rotation, magnification and shearing may be sufficient. However, if the regions of interest are large, including virtually an entire image, warped elastic transformations may be applied following the best rigid body registration.

One manner for implementing warped registration is the use of multi-scale, multi-region pyramidal approaches. In such approaches, a different cost function, entropy, mutual information, highlighting changes may be optimized at different scales. An image is re-sampled at a given scale, and then divided into multiple regions. Separate shift vectors are calculated at different regions. Shift vectors are interpolated to produce a smooth shift transformation, which is applied to warp the image. The image is re-sampled and the warped registration process is repeated at the next higher scale until the desired higher scale is reached.

Shape-Based Analysis: Large classes of objects can be decomposed into characteristics that assign local shape properties to shape attributes. Anatomies and features of interest may exhibit shared characteristics that permit their association in this manner. For example, vessels may be imaged and classified as having a generally cylindrical shape, whereas tumors may be substantially spherical. Colon Lumen exhibits ridges or valleys. Specific types of tumors may exhibit elongated structures, or specific surface characteristics.

Geometrical 3D filtering may be used to estimate local shape primitives, such as minimum and maximum curvatures, of image data so as to assign shape characteristics to anatomical objects. By way of example, shape-based methods may be used to identify vessels as cylinders in the arterial phase and hepatocellular carcinoma as spheres in the delayed arterial phase of a multi-phase contrast enhanced CT exam of the abdomen. Filter responses can be displayed on any view (sagittal, coronal or axial) as well as on a volume rendered view. In certain techniques, the user has the ability to selectively choose which type of response from a set of spherical, cylindrical or other responses they desire.

Exemplary Contrast Agent Imaging-Driven Paths: Based upon the foregoing processing and analysis techniques, and upon image data acquired for specific tissues and anatomies, with the image data being one of the types described above, the paths set forth in FIG. 5 may be performed to render various novel types of health care. As examples of the exemplary paths, the present discussion relates to health care for the liver in particular. As will be appreciated by those skilled in the art, the liver is the primary blood filtration and detoxification organ of the body. Moreover, it produces important enzymes in the form of bile that aide in the digestive processes of the body. Blood is supplied to the liver from two sources. Approximately 25% of the blood supplied to the liver originates in the arterial network, while the majority of the blood supplied, approximately 75%, originates in the hepatic veins. Functioning of the liver can be degraded or even halted by various diseases, such as cirrhosis and hepatitis, and by cancers. Primary cancer of the liver, hepatocellular carcinoma (HCC) is the eighth most common malignancy in the world. HCC is more common in the developing world with an incidence of 90/10,000 v. 2.4/100,000 for the United States. The disease typically manifests itself with attachments to the arterial network of the liver. The liver is also the sight for most metastatic cancers originating in other organs (lung, colon, breast, etc.). This is largely due to the blood filtration function of the liver.

Tumors in the liver are hypo-dense, making their detection difficult using standard CT imaging techniques. In existing techniques, a contrast agent is injected and a subsequent CT scan is performed. However, the different location and nutrient supply of HCC tumors as opposed to metastatic tumors has heretofore made detection of both cancers in a single imaging sequence impossible.

By the present technique, such detection, and even segmentation, sizing, and other processes are available. In particular, in the case of HCCs and metastatic tumors, it has been realized by the inventors that HCCs appear contrasted to the remainder of the liver parenchyma during the arterial phase of contrast agent progression, whereas metastatic tumors become contrasted in the portal venous phase of contrast agent progression. The portal venous phase corresponds to circulation of the agent back through the abdomen. While existing techniques generally permit only imaging hepatic venous phase contrast agent progression, the present techniques permit imaging in multiple phases. Employing high speed CT image acquisition, the present technique allows for imaging of multiple structures that would not be visible or contrasted by heretofore know processing techniques.

In an exemplary implementation, and still applied to liver health, the present technique foresees a high speed CT scan performed during an arterial phase of contrast agent progression, resulting in good contrast of arteries. A second high speed CT scan is then performed at a delayed arterial phase, permitting contrast of HCCs as well as three-dimensional images of the liver. Finally, a third high speed CT scan can be performed during the portal venous phase of contrast agent progression to provide contrast for metastatic tumors.

As will be appreciated by those skilled in the art, image data may be collected at a single phase of contrast agent progression or at multiple phases as mentioned above. The phases may be reduced from three or more to a pair of phases, for example, such as by performing scans at a delayed arterial phase and a portal venous phase.

The resulting data may be analyzed, such as to identify and segment the tumors visible in the image data. This may be performed in automated or semi-automated fashions as mentioned above. Moreover, the image data may be registered to permit visualization of the entire liver or a portion of the liver, with liver tumors of different types as well as liver vasculature across multiple image data sets. Moreover, in accordance with the present techniques, such fusion of image data may be performed from the same or different modalities, and even over expansions of time. Similarly, such comparisons may be made between multiple patients. Moreover, delineation of the liver into its segments may be performed. As will be appreciated by those skilled in the art, such delineation may provide an estimate of the volume of those segments. Such volume estimates may be used for advising caretakers prior to surgery, or even to provide information relating to possible sizing and compatibility for implantations. Similarly, three-dimensional visualization and modeling of the anatomy of interest is possible for planning surgical procedures. Such procedures may include radical procedures, such as transplantation, or minimally invasive procedures, such as radio frequency ablation, resections, cryoablation, and so forth. Similarly, real-time or near real-time visualization may be performed for surgical procedures as discussed above.

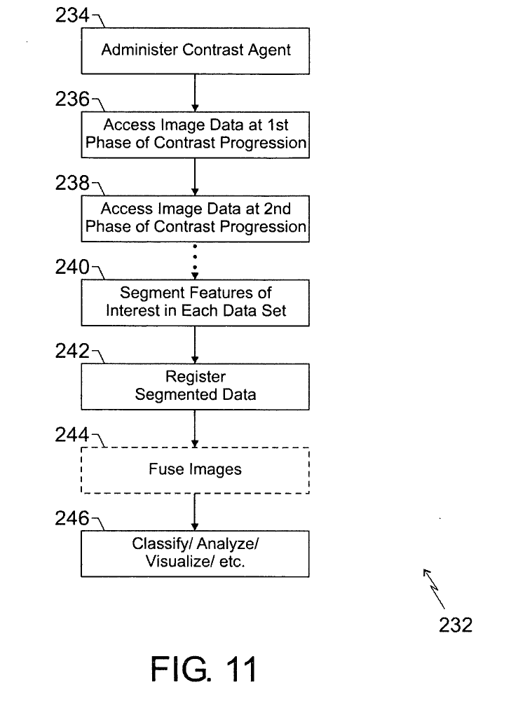

An exemplary workflow of process for such liver imaging, and indeed imaging of other organs and tissues in accordance with the present technique is provided in FIG. 11. The workflow, denoted by reference numeral 232, generally follows the workflow set forth in FIG. 3 above, but in greater detail for the particular process envisioned. The workflow begins at step 234 where a contrast agent is administered. The contrast agent may be any suitable contrast agent including those presently employed in the art, and may be administered in any suitable known manner. At step 236 image data is acquired at a first phase of contrast agent progression. As noted above, in the case of liver imaging via a high speed CT system, this step may be performed during a delayed arterial phase of contrast agent progression. The data from the acquisition is stored and may be partially or fully processed by the imaging system. In certain cases, it may be advantageous to store the raw or processed data for later image reconstruction and analysis. At step 238, image data is then acquired at a second phase of contrast agent progression. Again, in the example discussed above, this step may be performed during a portal venous phase of contrast agent progression, and the image data again either stored in raw form or processed immediately. Additional phases of contrast agent progression may be subject to additional image data acquisition following step 238.

As will be appreciated by those skilled in the art, in the example provided in FIG. 11, image data is collected during a single examination, as contrast agent progresses through the patient. Similar techniques may, of course, be performed for image data collected at different points in time, or on different subjects. Similarly, the steps may be performed on different imaging systems, including systems of different modalities. Finally, the steps may be performed at different system settings, permitting the viewing and analysis of tissues that are differentially contrasted due to the presence of the contrast agent.

Returning to the example of liver health care, at step 240 in FIG. 11, features of interest may be segmented from each data set. Particularly of interest will be vasculature of the liver, HCCs and metastatic tumors. Also particularly of interest may be segments of the liver. The segmented data may be registered as indicated at step 242, thereby co-locating the features of interest in a manner that provides additional detail and information for the health care provider. Optionally, at step 244, the images may be fused so as to create a composite image permitting viewing and analysis of all of the features of interest, or particular features of interest viewable in the separate data sets. Finally, at step 246, the features may be classified, analyzed, visualized or otherwise processed. As will be appreciated by those skilled in the art, the analysis and classification may be implemented by various computer-aided processing techniques, some of which are sometimes referred to as computer-aided detection or computer-aided diagnosis algorithms. Such algorithms generally compare features and characteristics of the anatomies that are segmented from the image data with known pathologies and anatomical characteristics. Based upon such comparisons, automated or semi-automated classifications can be made. These classifications can then be reviewed by specialists, to confirm or correct the reading.

FIG. 12 represents the collection of image data in the process of FIG. 11 at different points in time as contrast agent progresses through a patient. Thus, as indicated at reference numeral 248, a first scanning sequence may be performed at a first point in time t1 to collect image data as the contrast agent progresses through the patient in a first phase. As indicated at reference numeral 250, at a second point in time t2, an additional scan is performed to collect additional image data. Finally, at a third point in time, as indicated by reference numeral 252, additional image data may be collected. As will be appreciated by those skilled in the art, and as discussed above, the use of a plurality of points in time for scanning is particularly useful where anatomies and features of interest are not visible or are insufficiently visible at particular phases, permitting different visualization of multiple different anatomies and visualization of the anatomies separately.

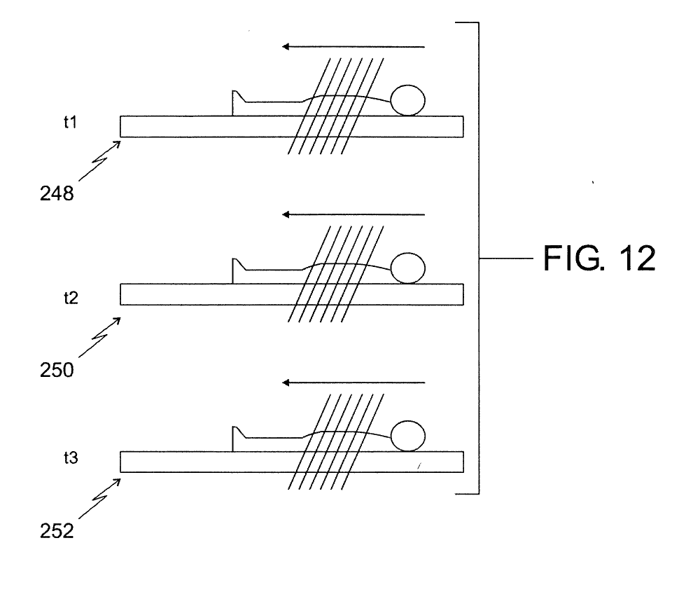

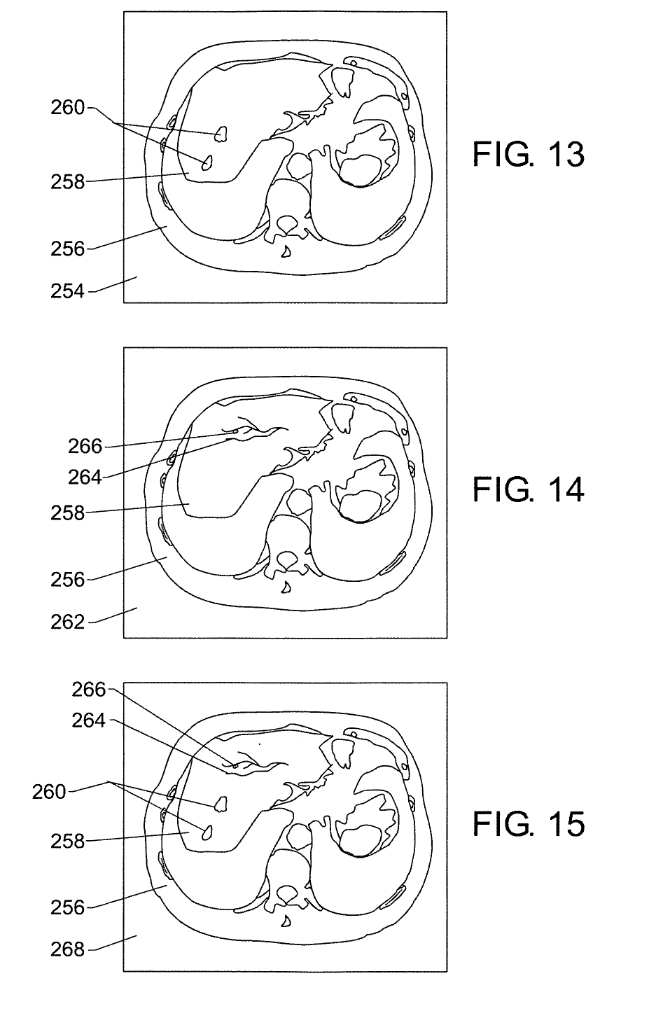

FIG. 13 represents an exemplary CT image 254 of the type that may result from reconstruction of image data acquired and analyze as discussed above. The image 254 is made based on high speed CT data acquisition during a portal venous phase of contrast agent progression. Accordingly, the image records the internal organs and tissues of the patient, as denoted generally by reference numeral 256, and particularly permits analysis of the liver 258. Due to the supply of nutrients to metastatic tumors by the venous blood, these tumors may be contrasted in the image 254, as indicated at reference numeral 260. FIG. 14, on the other hand, represents reconstructed image 262 made based upon image data taken during a delayed arterial phase of contrast agent progression. Again, the internal features of the patient are visible, as is the liver 258. Here, however, vasculature 264 becomes contrasted, along with HCCs 266 due to their close association with the arterial blood flow. FIG. 15 represents a fuse image in which the features of both images 254 and 262 are included. Such composite images 268 may be formed and saved as a separate data sets, or may be created as layers, permitting a health care provider to view one or the other of specific anatomies or all of the anatomies selectively.

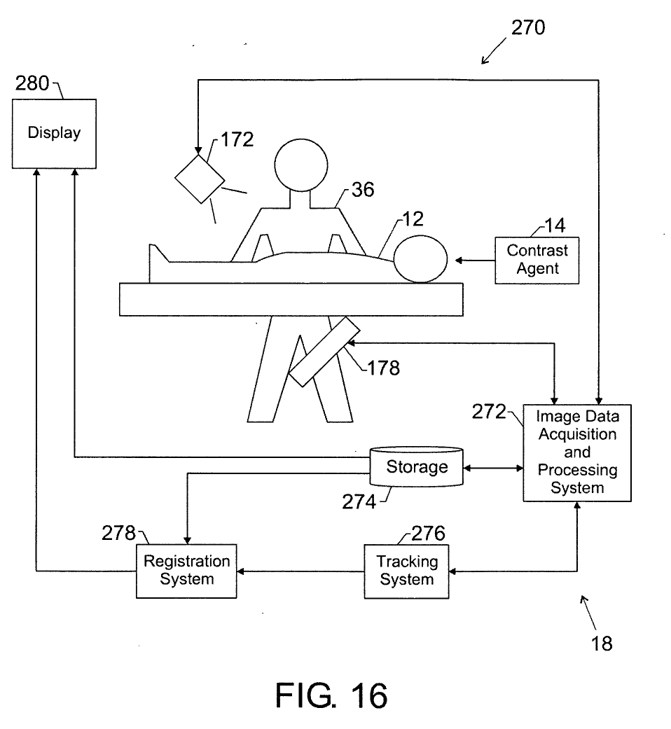

As noted above, the present techniques may be used for a wide range of tissues, analyses and procedures, as well as for range of purposes, such as medical treatment. FIG. 16 illustrates an exemplary installation for image-guided surgery according to the present techniques. In the illustration of FIG. 16, the installation is designated by reference numeral 270, and is illustrated as a surgical procedure being carried out by a medical care professional 36 on a patient 12. The patient is positioned in an imaging system, in this case including a radiation source 72 and a digital detector 78 of the type described above, such as for a digital X-ray system. A contrast agent 14 is administered to produce the desired contrast in the tissues on which the surgery is performed. The imaging system components are linked to an image data acquisition and processing system 272 which may include, for example, X-ray systems, fluoroscopy systems, ultrasound systems, and so forth.

The system 272 may draw upon and store image data in a storage unit 274, either within the system or remote from the system, such as in a hospital PACS. The system 272 will typically incorporate a tracking system 276 that permits surgical instruments, anatomical features, and so forth to be tracked via image data produced and processed by system 272. A registration system 278 can register the tracked components on patient images such that the surgeon can determine where and with respect to what anatomies the components are positioned. In a typical insulation, the components may include surgical probes, surgical instruments, catheters, stents, and so forth to mention only a few. Data produced by the imaging system, the tracking system and the registration system may be displayed in real-time or near real-time on a display 280. As noted above, the use of contrast agent and particular imaging techniques in accordance with the present implementations permits real-time surgical operations to be performed on anatomies and organs that could not be possible through heretofore known techniques.

While only certain features of the invention have been illustrated and described herein, many modifications and changes will occur to those skilled in the art. It is, therefore, to be understood that the appended claims are intended to cover all such modifications and changes as fall within the true spirit of the invention.

1. A method for evaluating health of liver tissue comprising:
accessing image data encoding contrast produced as a result of administration of a contrast agent for tissue of the liver during at least two phases of progression of the contrast agent; and
processing the image data to analyze features of interest in the tissue.

2. The method of claim 1, wherein the image data is acquired during at least two of an arterial phase, a delayed arterial phase and a portal venous phase of contrast agent progression.

3. The method of claim 1 wherein the image data is processed to identify hepatocellular cancers or metastatic tumors in the tissue.

4. The method of claim 1, wherein the accessed image data is produced via a high speed computed tomography scanning sequence.

5. The method of claim 1, wherein at least one diagnostic image is generated based upon the accessed image data.

6. The method of claim 5, wherein the at least one diagnostic image includes features discernable during each of the at least two phases of contrast agent progression.

7. A method for evaluating health of liver tissue comprising:
accessing image data encoding contrast produced as a result of administration of a contrast agent for tissue of the liver during an arterial or delayed arterial phase of progression of the contrast agent; and
processing the image data to analyze features of interest in the tissue.

8. The method of claim 7, comprising accessing image data for the tissue of the liver during at least one additional phase of progression of contrast agent and processing the image data to analyze additional features of interest in the tissue.

9. The method of claim 7, wherein the feature of interest includes hepatocellular carcinomas.

10. The method of claim 7, wherein the accessed image data is produced via a high speed computed tomography scanning sequence.

11. The method of claim 7, wherein at least one diagnostic image is generated based upon the accessed image data.

12. A method for evaluating health of tissue comprising:
accessing image data encoding contrast produced as a result of administration of a contrast agent to at least one subject to produce data for an image of liver tissue, fat tissue, vascular tissue, tumor tissue, stones, kidney tissue or pancreas tissue, the data including multi-phase scan data, temporal data, multi-modal data or multi-patient data; and
analyzing the data via a segmentation algorithm adapted for detection of the tissue, diagnosis of disease of the tissue, establishment of a care plan for medical treatment of the tissue, medical treatment of the tissue, transplantation of replacement tissue, follow-up on treatment of the tissue, analysis of health of the tissue or image guided surgery on the tissue.

13. A method for evaluating health of tissue comprising:
accessing image data encoding contrast produced as a result of administration of a contrast agent to at least one subject to produce data for an image of liver tissue, fat tissue, vascular tissue, tumor tissue, stones, kidney tissue or pancreas tissue, the data including multi-phase scan data, temporal data, multi-modal data or multi-patient data; and
analyzing the data via a sizing algorithm, the algorithm being adapted for detection of health of the tissue, diagnosis of disease of the tissue, establishment of a care plan for medical treatment of the tissue, medical treatment of the tissue; transplantation of replacement tissue, follow-up on treatment of the tissue, or analysis of health of the tissue.

14. A method for evaluating health of tissue comprising:
accessing image data encoding contrast produced as a result of administration of a contrast agent to at least one subject to produce data for an image of liver tissue, fat tissue, vascular tissue, tumor tissue, stones, kidney tissue or pancreas tissue, the data including multi-phase scan data, temporal data, multi-modal data or multi-patient data; and
analyzing the data via a visualization algorithm, the algorithm being adapted for detection of health of the tissue, establishment of a care plan for medical treatment of the tissue, transplantation of replacement tissue, follow-up on treatment of the tissue, analysis of health of the tissue or image guided surgery on the tissue.

15. A method for evaluating health of tissue comprising:
accessing image data encoding contrast produced as a result of administration of a contrast agent to at least one subject to produce data for an image of liver tissue, fat tissue, vascular tissue, tumor tissue, stones, kidney tissue or pancreas tissue, the data including multi-phase scan data, temporal data, perfusion data, or multi-modal data; and
analyzing the data via a registration algorithm, the algorithm being adapted for detection of health of the tissue, diagnosis of disease of the tissue, establishment of a care plan for medical treatment of the tissue, medical treatment of the tissue, transplantation of replacement tissue, follow-up on treatment of the tissue or performing image guided surgery on the tissue.

16. Method for evaluating health of tissue comprising:
accessing image data encoding contrast produced as a result of administration of a contrast agent to at least one subject to produce data for an image of vascular tissue, tumor tissue, stones, the data including single-phase scan data, multi-phase scan data, or multi-modal data; and
analyzing the data via a shape-based analysis algorithm, the algorithm being adapted for detection of the tissue, establishment of a care plan for medical treatment of the tissue or performing minimally invasive surgery on the tissue.

17. A method for evaluating health of tissue comprising:
accessing image data encoding contrast produced as a result of administration of a contrast agent to at least one subject to produce data for an image of liver tissue, vascular tissue, tumor tissue, kidney tissue or pancreas tissue, the data including multi-phase scan data, perfusion data, multi-modal data or multi-patient data; and
analyzing the data via a delineation algorithm, the algorithm being adapted for diagnosis of disease of the tissue, establishment of a care plan for medical treatment of the tissue, medical treatment of the tissue, transplantation of replacement tissue, analysis of health of the tissue or performing image guided surgery on the tissue.

18. A method for evaluating health of tissue comprising:
accessing image data encoding contrast produced as a result of administration of a contrast agent to at least one subject to produce data for an image of liver tissue, tumor tissue, stones, kidney tissue or pancreas tissue, the data including multi-phase scan data, temporal data, multi-modal data or multi-patient data; and
analyzing the data via a volumetric analysis algorithm, the algorithm being adapted for establishment of a care plan for medical treatment of the tissue, transplantation of replacement tissue, follow-up on treatment of the tissue, or analysis of health of the tissue.

19. A method for evaluating health of tissue comprising:
accessing image data encoding contrast produced as a result of administration of a contrast agent to at least one subject to produce data for an image of liver tissue, tumor tissue, stones, kidney tissue or pancreas tissue, the data including single-phase scan data, multi-phase scan data, temporal data, multi-modal data or multi-patient data; and
analyzing the data via a modeling algorithm, the algorithm being adapted for establishment of a care plan for medical treatment of the tissue, medical treatment of the tissue; transplantation of replacement tissue, analysis of health of the tissue or performing image guided surgery on the tissue.

20. A method for evaluating health of tissue comprising:
accessing image data encoding contrast produced as a result of administration of a contrast agent to at least one subject to produce data for an image of liver tissue, vascular tissue, tumor tissue, kidney tissue or pancreas tissue, the data including single-phase scan data, multi-phase scan data, multi-modal data; and
analyzing the data via a surgical navigation algorithm, the algorithm being adapted for treatment of the tissue or performing image guided surgery on the tissue.

21. A method for evaluating health of tissue comprising:
accessing image data encoding contrast produced as a result of administration of a contrast agent to at least one subject to produce data for an image of liver, the data including single-phase scan data, multi-phase scan data, or multi-modal data; and
analyzing the data via a texture-based analysis algorithm, the algorithm being adapted for detection of the tissue, establishment of a care plan for medical treatment of the tissue or performing minimally invasive surgery on the tissue.

22. A method for evaluating health of tissue comprising:
accessing image data for an image of liver tissue, the data including single modality scan data, or multi-modal data; and
analyzing the data via a texture-based analysis algorithm, the algorithm being adapted for detection of the tissue, establishment of a care plan for medical treatment of the tissue or performing minimally invasive surgery on the tissue.

23. A method for evaluating health of tissue comprising:
accessing image data acquired following administration of a contrast agent to at least one subject to produce data for an image of sascular tissue, tumor tissue, or stones, the data including single-phase scan data, multi-phase scan data, or multi-modal data; and
analyzing the data via a shape-based or texture-based analysis algorithm, the algorithm being adapted for detection of the tissue, establishment of a care plan for medical treatment of the tissue or performing minimally invasive surgery on the tissue.

24. A method for evaluating drug treatment for cancer, the method comprising:
accessing image data acquired following administration of a contrast agent to at least one subject;
delineating tissue suspect of disease;
registering tissue across multiple phases of contrast agent progression in the tissue; and
analyzing contract agent progression through the tissue to characterize drug treatment response.
